(19) United States
(12) Patent Application Publication
(10) Pub. No.: US 2024/0238176 A1
(43) Pub. Date: Jul. 18, 2024

(54) ANTI-POLLUTION ECO-FRIENDLY MATERIAL AND METHOD FOR PREPARING THE SAME

(71) Applicant: RESEARCH & BUSINESS FOUNDATION SUNGKYUNKWAN UNIVERSITY, Suwon-si (KR)

(72) Inventors: Younchul KIM, Seongnam-si (KR); Changsik SONG, Seoul (KR); Hwi Hyun MOON, Suwon-si (KR)

(73) Assignee: RESEARCH & BUSINESS FOUNDATION SUNGKYUNKWAN UNIVERSITY, Suwon-si (KR)

(21) Appl. No.: 18/401,930

(22) Filed: Jan. 2, 2024

(30) Foreign Application Priority Data
Jan. 2, 2023 (KR) 10-2023-0000436

(51) Int. Cl. A61K 8/29; A61K 8/02; A61Q 17/04; B01J 20/06; B01J 20/26; B01J 20/32

(52) U.S. Cl. CPC A61K 8/29; A61K 8/0275; A61Q 17/04; B01J 20/06; B01J 20/265; B01J 20/3204; B01J 20/3219; B01J 20/3274; B01J 20/3293; A61K 2800/614

(57) ABSTRACT

Disclosed are an anti-pollution cosmetic material and a method for preparing the same, wherein the anti-pollution cosmetic material contains a material having ultraviolet-blocking properties or heavy metal adsorption capacity or an organic-inorganic hybrid material having both ultraviolet-blocking properties and heavy metal adsorption capacity, so that the use of the inventive titanium dioxide (TiO₂) composite particles and heteroatomic polymer-attached polyamino acid polymer can provide not only an ultraviolet-blocking or anti-pollution cosmetic composition but also a cosmetic composition having both an ultraviolet-blocking effect and an anti-pollution effect, and such a material can mitigate the white cast and the harmfulness of a microparticle powder, which are problems of existing inorganic sunblocks, and can be utilized as an anti-pollution material for skin protection having heavy metal adsorption capacity.

### CROSS-REFERENCE TO RELATED APPLICATION (S)

This application is based on and claims priority under 35 U.S.C. 119 to Korean Patent Application No. 10-2023-0000436, filed on Jan. 2, 2023, in the Korean Intellectual Property Office, the disclosure of which is herein incorporated by reference in its entirety.

### BACKGROUND OF THE INVENTION

### 1. Field of the Invention

The present disclosure was established with the support of the Ministry of Trade, Industry and Energy, Republic of Korea, under Project No. 20013794, which was conducted by Sungkyunkwan University Industry-Academic Cooperation Foundation in the research project named “Center for Coupled Design Manufacturing of Composite Materials Parts” within the research program titled “Industrial Technology Base Center Fostering Pilot Project” under the management of the Korea Planning & Evaluation Institute of Industrial Technology, from 1 Jan. 2022 to 31 Dec. 2022.

This application claims priority and the benefit of Korean Patent Application No. 10-2023-0000436 filed in the Korean Intellectual Property Office on 2 Jan. 2023, the disclosure of which is incorporated herein by reference.

The present disclosure relates to an anti-pollution cosmetic material and a method for preparing the same and, specifically, to an anti-pollution cosmetic material comprising a material having ultraviolet-blocking properties or heavy metal adsorption capacity or an organic-inorganic hybrid material having both ultraviolet-blocking properties and heavy metal adsorption capacity, and to a method for preparing the same.

### 2. Description of the Prior Art

Recently, interest in ultraviolet blocking has been rapidly increasing due to ozone layer destruction and global warming resulting from environmental problems. In particular, ultraviolet light may cause rapid aging, wrinkling, and pigmentation of the human skin, and thus various screens are being developed.

Sunblocks are largely classified into inorganic sunblocks, which are physical sunblocks, and organic sunblocks, which are chemical sunblocks. Titanium dioxide (TiO₂) and Zinc oxide (ZnO), which are used as typical inorganic sunblocks, have the advantage of having high skin stability and effectively blocking UVA and UVB. However, inorganic sunblocks scatter visible light as well as ultraviolet light to cause a white cast on the skin, and the presence of TiO₂ causes harm to humans by a nanoparticle powder present in products. On the other hand, organic sunblocks, which mainly employ aromatic substances, such as avobenzone or oxybenzone, have the advantage of absorbing ultraviolet light to protect the skin from ultraviolet light and causing no white cast to secure transparency, but has the disadvantage of having poor light stability and being toxic and irritating to the skin of the human body.

Recently, fine dust in the atmosphere causes various health problems and, particularly, the human body is exposed to various diseases due to heavy metals in fine dust, including respiratory diseases.

Heavy metals contained in fine dust or yellow dust attach to or penetrate into the skin to cause problems such as skin troubles, thereby accelerating urban skin aging, and therefore, interest in anti-pollution materials that effectively protect the skin from harmful factors in urban environments and block the penetration of heavy metals into the skin is increasing.

### PRIOR ART DOCUMENTS

### Patent Documents

(Patent Document 01) Korean Patent No. 10-1854855

(Patent Document 02) Korean Patent Publication No. 10-2015-0151865

### SUMMARY OF THE INVENTION

The present inventors have made intensive research efforts to develop a material having ultraviolet-blocking properties or heavy metal adsorption capacity or a novel organic-inorganic hybrid material having both ultraviolet-blocking properties and heavy metal adsorption capacity. As a result, the present inventors established that a novel organic-inorganic hybrid material having both ultraviolet-blocking properties and heavy metal adsorption capacity, prepared by fusing a titanium dioxide (TiO₂) derivative having ultraviolet-blocking properties and a biocompatible polymer having heavy metal adsorption capacity, had an excellent effect in ultraviolet blocking and/or heavy metal adsorption, and thus completed the present disclosure.

Accordingly, an aspect of the present disclosure is to provide a material having ultraviolet-blocking properties.

Another aspect of the present disclosure is to provide a material having heavy metal adsorption capacity.

Still another aspect of the present disclosure is to provide an organic-inorganic hybrid material having both ultraviolet-blocking properties and heavy metal adsorption capacity.

The present inventors have made intensive research efforts to develop a material having ultraviolet-blocking properties or heavy metal adsorption capacity or a novel organic-inorganic hybrid material having both ultraviolet protection characteristics and heavy metal adsorption capacity. As a result, the present inventors established that a novel organic-inorganic hybrid material having both ultraviolet-blocking properties and heavy metal adsorption capacity, prepared by fusing a titanium dioxide (TiO₂) derivative having ultraviolet-blocking properties and a biocompatible polymer having heavy metal adsorption capacity, had an excellent effect in ultraviolet blocking and/or heavy metal adsorption.

In accordance with an aspect of the present disclosure, there is provided a method for preparing titanium dioxide composite particles, the method comprising:
(a) preparing a titanium dioxide (TiO₂) derivative with an amine (−NH₂) functional group introduced to the surface of titanium dioxide (TiO₂); and
(b) reacting the titanium dioxide (TiO₂) derivative obtained in step (a) and a polyamino acid derivative to fuse the titanium oxide derivative and the polyamino acid derivative.

The titanium dioxide composite particles of the present disclosure are prepared by reaction of the amine functional group introduced to the surface of titanium dioxide (TiO₂) with the polyamino acid derivative, and specifically, by fusing through a ring-opening addition reaction of the amine functional group introduced to the surface of titanium dioxide (TiO₂) with the polyamino acid derivative.

As used herein, the term “titanium dioxide” refers to an oxide of titanium, represented by TiO₂, and a substance used as a sunblock for blocking ultraviolet light in a cosmetic composition, and as a typical example, an inorganic sunblock reflecting, scattering, and absorbing light.

Although having an excellent ultraviolet-blocking effect, titanium dioxide may cause a noticeable white cast due to its high refractive index when applied to the skin, and may cause the denaturation of formulation components and the pigmentation due to its high photo-catalytic ability to decompose or denature organic materials, especially pigments, under light energy. Furthermore, a cosmetic composition containing titanium dioxide may cause skin irritation.

It was identified in the examples of the present disclosure that titanium dioxide composite particles obtained by fusing a polyamino acid derivative and titanium dioxide with an amine functional group introduced to the surface thereof are materials having ultraviolet-blocking properties and, specifically, the material exhibited an ultraviolet-blocking effect at a similar level to powdery titanium dioxide, showed a reduced white cast when applied to the skin, and also suppressed photo-catalytic activity.

Recently, interest in respiratory diseases and fine dust reduction is increasing due to increasing fine dust in Korea. Particularly, the fine dust contains a large amount of harmful substances, such as heavy metals, and thus when penetrating deep into the skin, the fine dust may accelerate skin aging and provoke an inflammatory reaction, causing skin diseases. Therefore, the market for anti-pollution cosmetic products that protect the skin from external harmful environments has been on the rise.

In an embodiment of the present disclosure, the method for preparing titanium dioxide composite particles may further comprise, before step (b), attaching a heteroatomic compound to the polyamino acid derivative.

It was identified in an example of the present disclosure that the further comprising of attaching a heteroatomic compound to the polyamino acid derivative before step (b) could lead to a heavy metal adsorption effect as well as an ultraviolet-blocking effect along with a reduced white cast and a photo-catalytic activity suppressing effect.

In an embodiment of the present disclosure, the titanium dioxide (TiO₂) derivative with the amine functional group in step (a) may be formed by reaction of titanium dioxide and an aminosilane at a weight ratio of 1:1 to 1:5.

Therefore, the amine group introduced to the surface of titanium dioxide allows for the preparation of titanium dioxide composite particles having ultraviolet-blocking properties by reaction with the polyamino acid derivative.

As used herein, the term “polyamino acid derivative” refers to a polymer having a similar chemical structure to a protein and formed by (condensation) polymerization of a plurality of amino acids, and is distinguished from a polypeptide obtained by linking amino acids in a predetermined arrangement.

In an embodiment of the present disclosure, the polyamino acid derivative may be one selected from the group consisting of polysuccinimide (PSI), polyaspartic acid (PASA), polyaspartamide (PAA), and polyhydroxyethyl aspartamide (PHEA), and the polyamino acid derivative can be arbitrarily modified depending on amino acid monomers and is not particularly limited but, more specifically, the polyamino acid derivative may be polysuccinimide, but is not limited thereto.

In another embodiment of the present disclosure, the polyamino acid derivative may have a heteroatomic compound further attached thereto.

In the present disclosure, the heteroatomic compound may be at least one selected from the group consisting of histamine, aminoimidazole, aminomethylimidazole, aminopropylimidazole, aminomethylbenzimidazole, and histidine, specifically, histamine, but is not limited thereto.

As used herein, the term “heteroatomic compound” refers to a compound comprising an atom of any element, specifically, nitrogen, oxygen, sulfur, phosphorus, selenium, or the like, other than carbon or hydrogen, wherein the heteroatomic compound can be combined with metal ions to enable the adsorption of heavy metals.

A heteroatomic compound capable of adsorbing heavy metals is attached to the polyamino acid derivative to prepare a biocompatible type of material for adsorbing heavy metals, thereby providing a material exhibiting a heavy metal adsorption effect.

Furthermore, in the method for preparing titanium dioxide composite particles of the present disclosure, the heteroatomic compound is further attached to the polyamino acid derivative, and then the resultant product is fused to a titanium dioxide amine derivative, thereby providing a material exhibiting a heavy metal adsorption effect as well as an ultraviolet-blocking effect.

According to another aspect of the present disclosure, there is provided a titanium dioxide composite particles represented by Chemical Formula 1 below:

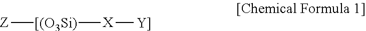

Z is a titanium dioxide particle;
X is a C₁ to C₆ linker; and
Y is

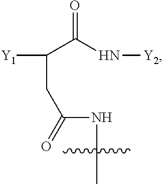

where Y₁ and Y₂ each are independently Compound A, Compound B, or a copolymer of Compound A and Compound B,
wherein Compound A is

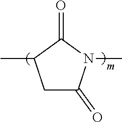

### and Compound B is

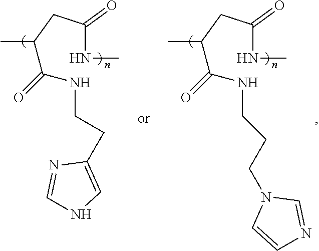

where m and n each are independently an integer of 1 to 1500.

In a specific embodiment of the present disclosure, m and n each may be independently an integer of 1 to 1500, 1 to 1200, 1 to 1000, 1 to 900, 1 to 800, 1 to 700, 1 to 600, 1 to 500, 1 to 400, 1 to 300, 1 to 200, or 1 to 100, but is not limited thereto.

In an embodiment of the present disclosure, Compound A may be prepared by the following reaction:

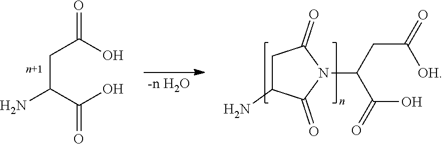

In an embodiment of the present disclosure, the molecular weight of Compound A, Compound B, or a copolymer of Compounds A and B may be up to 150 kDa.

In an embodiment of the present disclosure, the titanium dioxide composite particle may be characterized in that in the copolymer of Compounds A and B, Compound A and Compound B are present in a random sequence.

In another embodiment of the present disclosure, the titanium dioxide composite particle may be characterized in that in the copolymer of Compound A and Compound B, Compound A and Compound B are present in an alternating sequence.

In an embodiment of the present disclosure, the titanium dioxide composite particle may be characterized in that Y has the following structure:

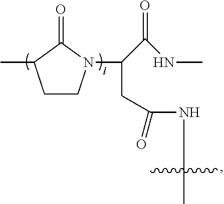

where 1 is an integer of 1 to 1500.

In a specific embodiment of the present disclosure, 1 may be an integer of 1 to 1500, 1 to 1200, 1 to 1000, 1 to 900, 1 to 800, 1 to 700, 1 to 600, 1 to 500, 1 to 400, 1 to 300, 1 to 200, or 1 to 100, but is not limited thereto.

In another embodiment of the present disclosure, the titanium dioxide composite particle may be characterized in that Y has the following structure:

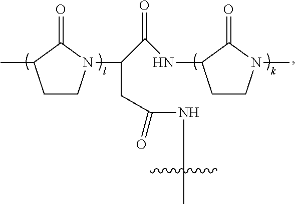

where l and k each are independently an integer of 1 to 800.

In a specific embodiment of the present disclosure, 1 and k may be an integer of 1 to 800, 1 to 700, 1 to 600, 1 to 500, 1 to 400, 1 to 300, 1 to 200, or 1 to 100, but are not limited thereto.

In an embodiment of the present disclosure, the titanium dioxide composite particle may be characterized in that Y has the following structure:

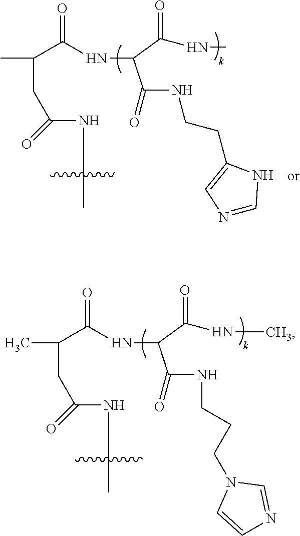

wherein k is an integer of 1 to 800.

In a specific embodiment of the present disclosure, k may be an integer of 1 to 800, 1 to 700, 1 to 600, 1 to 500, 1 to 400, 1 to 300, 1 to 200, or 1 to 100, but are not limited thereto.

In another embodiment of the present disclosure, the titanium dioxide composite particle may be characterized in that Y has the following structure:

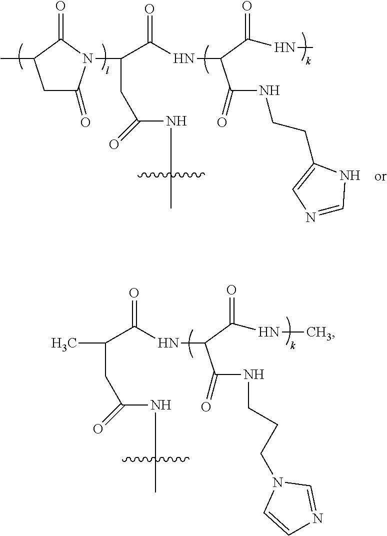

where l and k each are independently an integer of 1 to 800.

In a specific embodiment of the present disclosure, l and k may be an integer of 1 to 800, 1 to 700, 1 to 600, 1 to 500, 1 to 400, 1 to 300, 1 to 200, or 1 to 100, but are not limited thereto.

In accordance with still another aspect of the present disclosure, there is provided a polyamino acid polymer comprising Compound A and Compound B as monomers,
wherein Compound A is

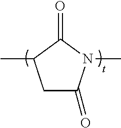

### and Compound B is

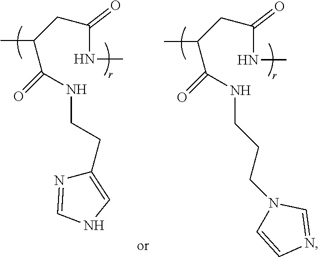

where t and r each are independently an integer of and 1 to 800.

In a specific embodiment of the present disclosure, t and r may be an integer of 1 to 800, 1 to 700, 1 to 600, 1 to 500, 1 to 400, 1 to 300, 1 to 200, or 1 to 100, but are not limited thereto.

In an embodiment of the present disclosure, the molecular weight of Compound A, Compound B, or a copolymer of Compounds A and B may be up to 150 kDa.

In an embodiment of the present disclosure, the polyamino acid polymer may be characterized in that Compound A and Compound B are present in a random sequence.

In another embodiment of the present disclosure, the polyamino acid polymer may be characterized in that Compound A and Compound B are present in an alternating sequence.

In accordance with still another aspect of the present disclosure, there is provided an ultraviolet-blocking cosmetic composition comprising titanium dioxide composite particles.

It was confirmed in the present disclosure that titanium dioxide composite particles, which may be obtained by fusing a titanium dioxide amine derivative and a polyamino acid polymer, had an ultraviolet-blocking effect, and therefore, a cosmetic composition comprising the particles can be expected to exhibit an ultraviolet-blocking effect.

In accordance with still another aspect of the present disclosure, there is provided an anti-pollution cosmetic composition comprising a polyamino acid polymer.

It was confirmed in the present disclosure that a biocompatible type of material for adsorbing heavy metals, which may be obtained by attaching a heteroatomic compound having heavy metal adsorption capacity to a polyamino acid polymer, had a heavy metal adsorbing effect, and therefore, a cosmetic composition comprising the material can be expected to exhibit an anti-pollution effect for the purpose of skin protection.

Furthermore, in the preparation of the titanium dioxide composite particles, (i) a heteroatomic compound is further attached to the polyamino acid polymer before the fusion of the titanium dioxide amine derivative and the polyamino acid polymer or (ii) the titanium dioxide amine derivative is fused to a polyamino acid polymer with a heteroatomic compound already attached thereto, so that the titanium dioxide composite particles can be predicted to exhibit not only an ultraviolet-blocking effect but also an anti-pollution effect of protecting the skin through heavy metal adsorption capacity by the heteroatomic compound.

The cosmetic composition may comprise common adjuvants, such as a stabilizer, a solubilizer, a vitamin, a pigment, and a flavor, and a carrier, in addition to the material according to the present disclosure as an active ingredient.

The cosmetic composition may be prepared in any formulation that is usually prepared in the art, and examples thereof may comprise a solution, a suspension, an emulsion, a paste, a gel, a cream, a lotion, a powder, an oil, a powder foundation, an emulsion foundation, a wax foundation, a spray, and the like, but are not limited thereto. More specifically, the cosmetic composition may be prepared in the formulation of a sun cream, an emollient lotion, an astringent lotion, a nourishing lotion, a nourishing cream, am essence, an eye cream, a pack, a spray, or a powder.

In cases where the formulation is a paste, cream, or gel, an animal oil, a vegetable oil, wax, paraffin, starch, tragacanth, a cellulose derivative, polyethylene glycol, silicone, bentonite, silica, talc, zinc oxide, or the like may be used as a carrier component.

In cases where the formulation is a powder or spray, lactose, talc, silica, aluminum hydroxide, calcium silicate, or a polyamide powder may be used as a carrier component, and particularly, in cases where the formulation is a spray, the spray may further comprise a propellant, such as chlorofluorohydrocarbon, propane/butane, or dimethyl ether.

In cases where the formulation is a solution or emulsion, a solvent, a solubilizer, or an emulsifier may be used as a carrier component, and examples thereof comprise water, ethanol, isopropanol, ethyl carbonate, ethyl acetate, benzyl alcohol, benzyl benzoate, propylene glycol, 1, 3-butyl glycol oil, a glycerol aliphatic ester, polyethylene glycol, or a sorbitan fatty acid ester.

In cases where the formulation is a suspension, a liquid diluent, such as water, ethanol, or propylene glycol, a suspension, such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier component.

Since the ultraviolet-blocking cosmetic composition and anti-pollution cosmetic composition of the present disclosure encompasses the method for preparing titanium dioxide composite particles, titanium dioxide composite particles, and a polyamino acid polymer according to other aspects of the present disclosure, the overlapping contents therebetween are recited and the description thereof will be omitted in order to avoid excessive complexity in the description of the present specification.

The use of the titanium dioxide (TiO₂) composite particles and the heteroatomic polymer-attached polyamino acid polymer of the present disclosure can provide not only an ultraviolet-blocking or anti-pollution cosmetic composition but also a cosmetic composition having both an ultraviolet-blocking effect and an anti-pollution effect. Furthermore, such a material can mitigate the white cast and the harmfulness of a microparticle powder, which are problems of existing inorganic sunblocks, and can be utilized as an anti-pollution material for skin protection having heavy metal adsorption capacity.

### BRIEF DESCRIPTION OF THE DRAWINGS

The above and other aspects, features and advantages of the present disclosure will be more apparent from the following detailed description taken in conjunction with the accompanying drawings.

FIG. 1 shows a titanium dioxide derivative with an amine group (T-NH₂) and titanium dioxide (TiO₂) composite particles (Material 1, T-PSI) having ultraviolet-blocking properties, which were prepared in Preparation Example 1-1.

FIG. 2 shows a biocompatible polyamino acid polymer material (Material 2, PH) having heavy metal adsorption capacity prepared in Preparation Example 1-2.

FIG. 3 shows titanium dioxide (TiO₂) composite particles (Material 3, T-PH) having both ultraviolet-blocking properties and heavy metal adsorption capacity prepared in Preparation Example 1-3.

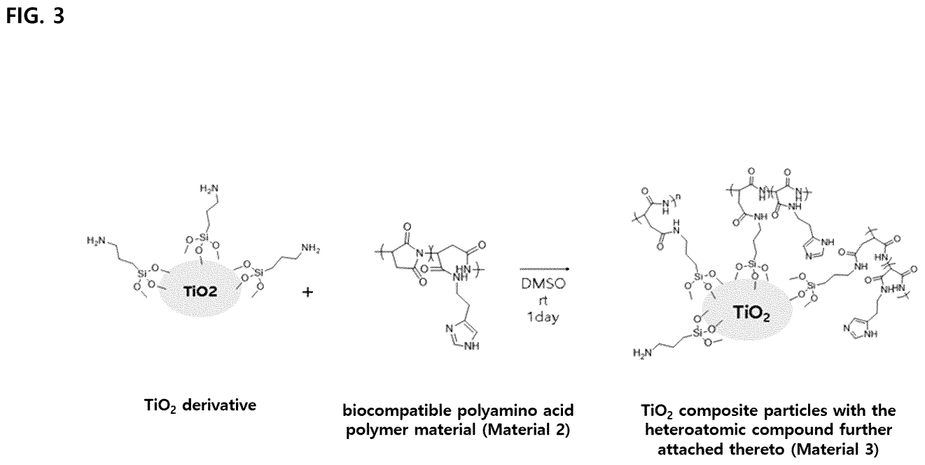

FIG. 4 shows a graph illustrating the infrared (FT-IR) spectroscopy analysis results of Materials 1 to 3 prepared in Preparation Examples 1-1 to 1-3.

FIG. 5 shows graphs illustrating the thermogravimetric analysis (TGA) results of Materials 1 to 3 prepared in Preparation Examples 1-1 to 1-3.

FIG. 6 shows a graph illustrating an NMR spectrum of Material 2 prepared in Preparation Example 1-2.

FIG. 7 shows (a) the composition of a water-in-oil (w/o) emulsion (b) a preparation method thereof in order to evaluate the ultraviolet-blocking effect.

FIG. 8 shows graphs illustrating the ultraviolet-blocking effects of Materials 1 and 3 prepared in Preparation Examples 1-1 and 1-3.

FIG. 9 shows graphs illustrating the heavy metal adsorption capacity of Material 2 prepared in Preparation Example 1-2.

FIG. 10 shows graphs illustrating the heavy metal adsorption capacity of Material 3 prepared in Preparation Example 1-3.

FIG. 11 shows a graph illustrating the photo-catalyst reducing effects of Materials 1 and 3 prepared in Preparation Examples 1-1 and 1-3.

FIG. 12 shows (a) a biocompatible polyamino acid polymer material (Material 4, PA) having a heavy adsorption capacity prepared in Preparation Example 2-1, and (b) a titanium dioxide (TiO₂) composite particles (Material 5, T-PA) having both ultraviolet-blocking properties and heavy metal adsorption capacity prepared in Preparation Example 2-2.

FIG. 13 shows a graph illustrating the NMR spectrum of Material 4 prepared in Preparation Example 2-1.

FIG. 14 shows a graph illustrating the results of infrared (FT-IR) spectroscopic analysis of Materials 1, 4 and 5 prepared in Preparation Example 1-1, Preparation Example 2-1, and Preparation Example 2-2, respectively.

FIG. 15 shows graphs illustration the results of thermogravimetric analysis (TGA) of Materials 1, 4 and 5 prepared in Preparation Example 1-1, Preparation Example 2-1, and Preparation Example 2-2, respectively.

FIG. 16 shows (a) the composition of a water-in-oil (w/o) emulsion (b) a preparation method thereof in order to evaluate the ultraviolet-blocking effect.

FIG. 17 shows graphs illustrating the ultraviolet-blocking effects of Materials 4 and 5 prepared in Preparation Example 2-1 and Preparation Example 2-2.

FIG. 18 shows graphs illustrating the heavy metal adsorption capacity of Material 4 prepared in Preparation Example 2-1.

FIG. 19 shows graphs illustrating the heavy metal adsorption capacity of Material 5 prepared in Preparation Example 2-2.

FIG. 20 shows shows a graph illustrating the photo-catalyst reducing effects of Materials 1 and 5 prepared in Preparation Examples 1-1 and 2-2.

### DETAILED DESCRIPTION OF THE EXEMPLARY EMBODIMENTS

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

### Preparation Examples 1

### Preparation Example 1-1. Preparation of Titanium Dioxide (TiO₂) Composite Particles (Material 1, T-PSI) Having Ultraviolet-Blocking Properties

### (1) Synthesis of Titanium Dioxide Derivative (T-NH₂)

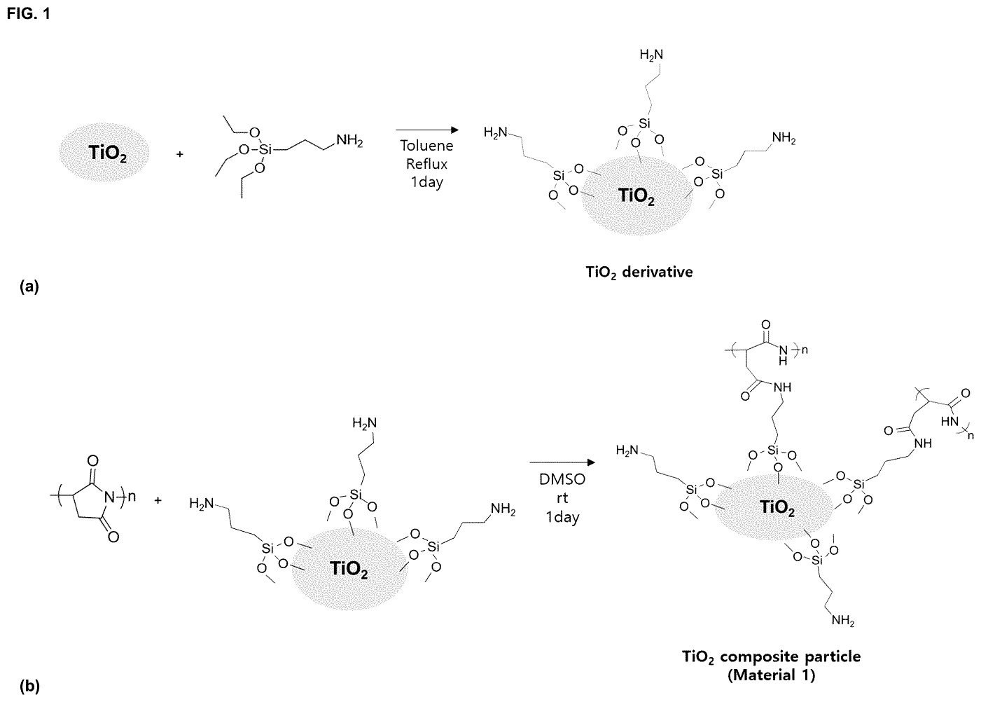

First, titanium dioxide (TiO₂) and an aminosilane were reacted to prepare a TiO₂ derivative with an amine (−NH₂) functional group introduced to the surface of titanium dioxide (TiO₂). A schematic diagram of the reaction is shown in (a) of FIG. 1.

Specifically, TiO₂ (1.5 g) was added into 50 ml of toluene under nitrogen conditions, followed by ultrasonic treatment for 30 min. Thereafter, 200 wt % of (3-aminopropyl) triethoxysilane (APTS) was added, followed by stirring in a reflux state at 150° C. for 3 days. The product was obtained by centrifugation, and the yield was 65.0%.

### (2) Synthesis of Titanium Dioxide (TiO₂) Composite Particles (Material 1, T-PSI)

Next, the titanium dioxide (TiO₂) derivative prepared in (1) above was reacted with polysuccinimide (PSI) belonging to a hydrophilic biodegradable polyamino acid polymer to produce titanium dioxide (TiO₂) composite particles (Material 1) having ultraviolet-blocking properties. A schematic diagram of the reaction is shown in (b) of FIG. 1.

Specifically, the TiO₂ derivative (2 g) modified with the amine group was added to 200 ml of DMSO, followed by ultrasonic treatment for 30 min. Thereafter, 100 wt % of polysuccinimide (PSI) was added, followed by stirring at room temperature for 2 days. The product was obtained by centrifugation, and the yield was 60.0%.

In order to conduct qualitative evaluation of the material prepared in the present disclosure, Fourier transform infrared analysis (FT-IR) and thermogravimetric analysis were performed.

Specifically, the Fourier infrared spectroscopy analysis was performed by a Vertex70 spectrometer (Bruker Optics, MA, USA) in the range of 600 cm⁻¹ to 4000 cm⁻¹, and the thermogravimetric analysis was performed by a TG/DTA7300 instrument (SEICO Instrument, Tokyo, Japan) from 0° C. to 800° C. with a temperature change of 10° C./min.

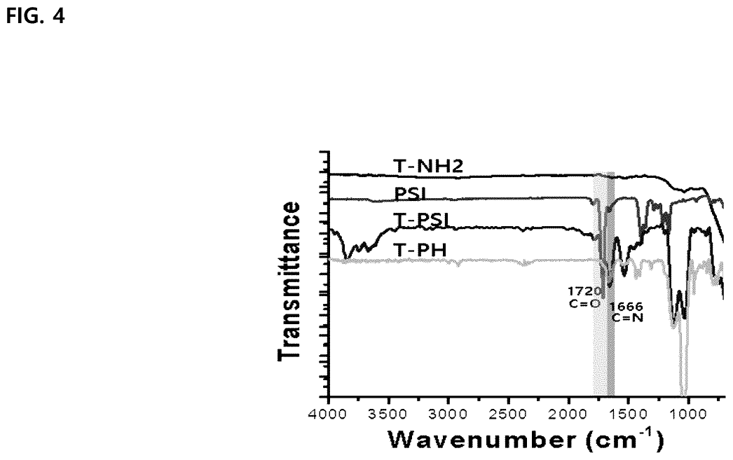

The FT-IR data results for Material 1 are shown in FIG. 4.

As shown in FIG. 4, the corresponding material was synthesized considering that an absorption peak of C═N (1666 cm⁻¹) of the histamine group was observed.

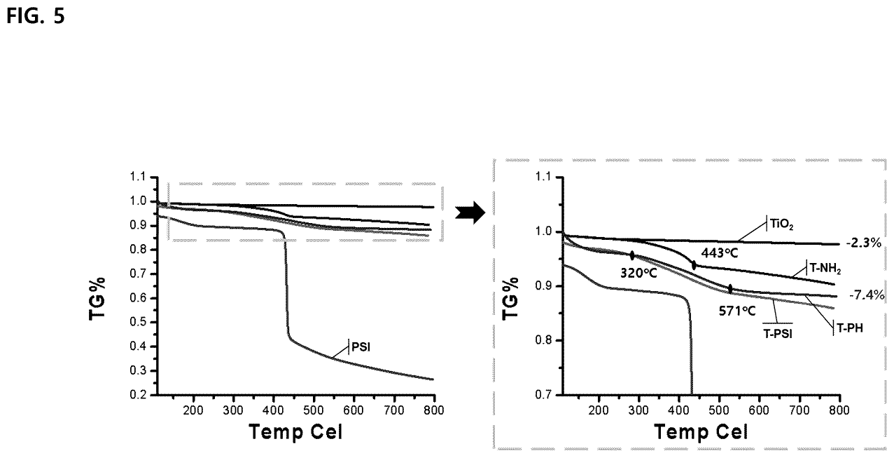

The TGA data for Material 1 are shown in FIG. 5.

As shown in FIG. 5, the thermal decomposition temperature of Material 1 (T-PSI) compared with TiO₂ was changed, indicating that the composite material was produced.

### Preparation Example 1-2. Preparation of Biocompatible Polyamino Acid Polymer Material (Material 2, PH) Having Heavy Metal Adsorption Capacity

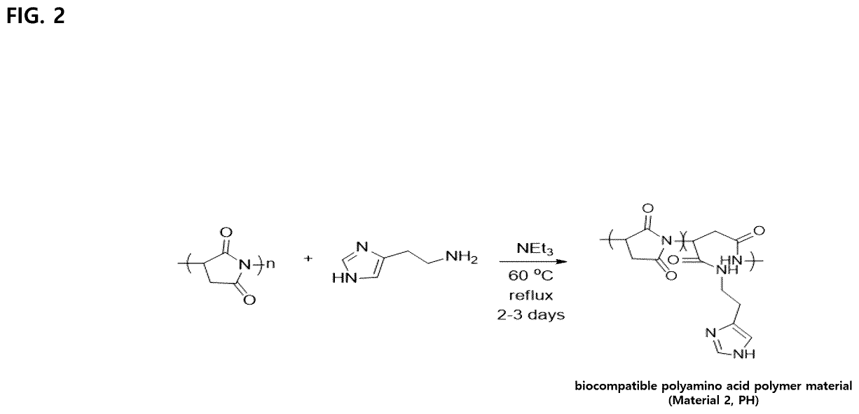

A biocompatible polyamino acid polymer material (Material 2) having heavy metal adsorption capacity was prepared by attaching a heteroatomic compound to polysuccinimide (PSI), a polyamino acid biocompatible polymer material. A schematic diagram of the reaction is shown in FIG. 2.

Specifically, PSI (2 g) was added into 25 ml of DMSO and dissolved with sufficient stirring. Thereafter, histamine dihydrochloride (0.92 g) and triethylamine (1.66 ml) were added, followed by stirring at room temperature for 2 days. Then, the product was obtained by drying through vacuum. The yield was 98%.

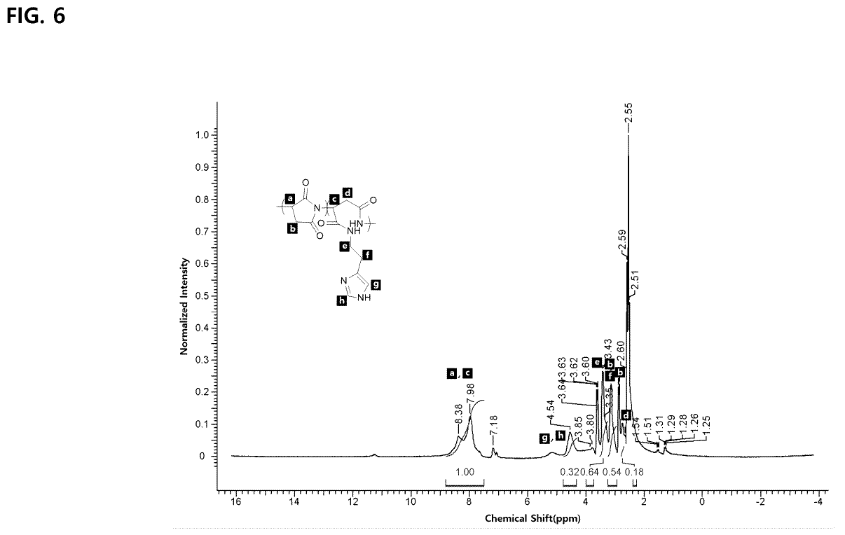

NMR data for Material 2 (PH) are shown in FIG. 6. FIG. 6 confirmed the synthesis of Material 2.

In addition, FT-IR data and TGA data for Material 2 (PH) are shown in FIGS. 4 and 5, respectively.

### Preparation Example 1-3. Preparation of Titanium Dioxide (TiO₂) Composite Particles (Material 3, T-PH) Having Ultraviolet-Blocking Properties and Heavy Metal Adsorption Capacity

The TiO₂ derivative (T-NH₂) of Preparation Example 1-1 and the biocompatible polyamino acid polymer material (PH) having heavy metal adsorption capacity of Preparation Example 1-2 were reacted to produce titanium dioxide (TiO₂) composite particles (Material 3, T-PH) having both ultraviolet-blocking properties and heavy metal adsorption capacity.

Specifically, the TiO₂ derivate (2 g) modified with the amine group was added to 40 ml of DMSO, followed by ultrasonic treatment for 30 min. Thereafter, 100 wt % of PH (Material 2) was added, followed by stirring at room temperature for 2 days. The product was obtained by centrifugation, and the yield was 64.0%.

FT-IR data and TGA data for Material 3 (T-PH) are shown in FIGS. 4 and 5, respectively.

### Test Examples 1

### Test Example 1-1. Verification of Ultraviolet-Blocking Effect

To verify the UV blocking effects of the titanium dioxide (TiO₂) composite particles (Material 1, T-PSI) prepared in Preparation Example 1-1 and the titanium dioxide (TiO₂) composite particles with the heteroatomic compound further attached thereto (Material 3, T-PH), UV-Vis spectrum analysis was performed to evaluate the absorbance and transmittance.

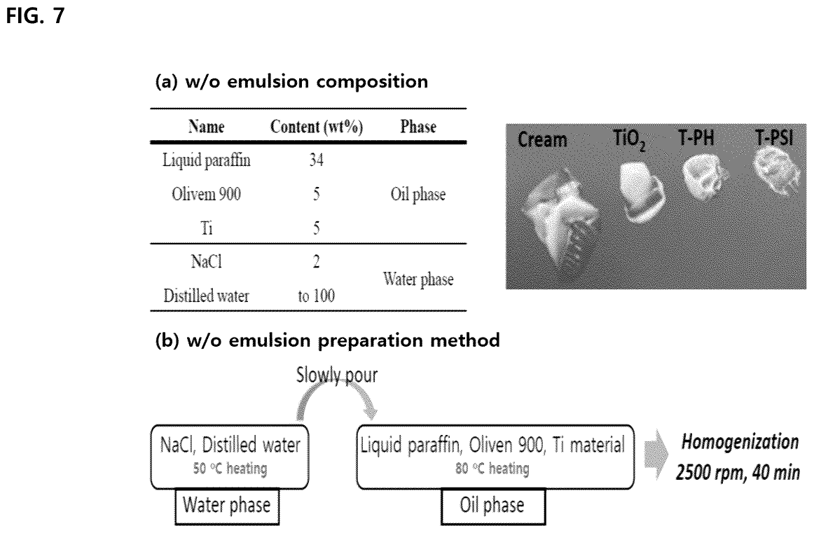

In this example, powdery TiO₂ confirmed to have an ultraviolet-blocking effect was used as a control group. By using (a) the composition of the water-in-oil (w/o) emulsion and (b) the preparation method thereof as shown in FIG. 7, each material was coated in a uniformly mixed cream form and was coated in a cream form on a polymethyl methacrylate (PMMA) plate to evaluate the ultraviolet-blocking effect.

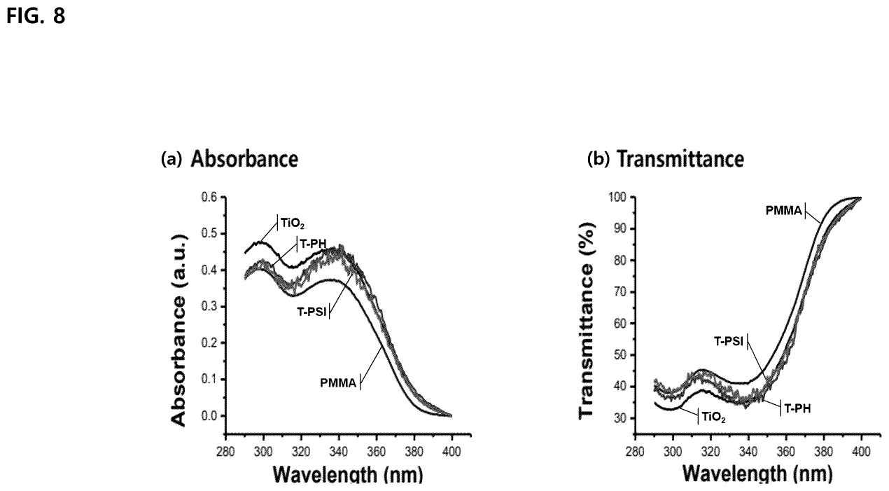

The results are shown (a) and (b) of FIG. 8.

As shown in (a) and (b) of FIG. 8, Material 1 (T-PSI) and Material 3 (T-PH) of the present disclosure exhibited an ultraviolet-blocking effect at a similar level to the powdery TiO₂ used as a control group.

### Test Example 1-2. Verification of Heavy Metal Adsorption Effect

To verify the heavy metal adsorption effect of the biocompatible polyamino acid polymer material (Material 2, PH) prepared in Preparation Example 1-2 and the titanium dioxide (TiO₂) composite particles with the heteroatomic compound further attached thereto (Material 3, T-PH), inductively coupled plasma (ICP) analysis was performed.

Specifically, Material 2 (PH) was exposed to copper (Cu²⁺) and nickel (Ni²⁺) aqueous solutions for 5 min, 30 min, and 2 h, respectively, and the adsorbed materials were re-precipitated in methanol (MeOH) two times. After that, the amounts of heavy metals adsorbed were measured to verify the heavy metal adsorption capacity. To measure the degrees of adsorption of copper (Cu²⁺) and nickel (Ni²⁺) depending on three concentrations (50,000 mg/L, 100,000 mg/L, and 300,000 mg/L), Material 2 was exposed to metal solutions with the three concentrations for 12 h, and then the adsorbed materials were re-precipitated in methanol two times to re-elute un-adsorbed heavy metals. After that, the amounts of heavy metals adsorbed were measured to verify the heavy metal adsorption capacity.

Material 3 was exposed to copper (Cu²⁺) and nickel (Ni²⁺) aqueous solutions for 12 h, and then the adsorbed materials were washed with water three times to re-elute un-adsorbed heavy metals. After that, the amounts of heavy metals adsorbed were measured to verify the heavy metal adsorption capacity.

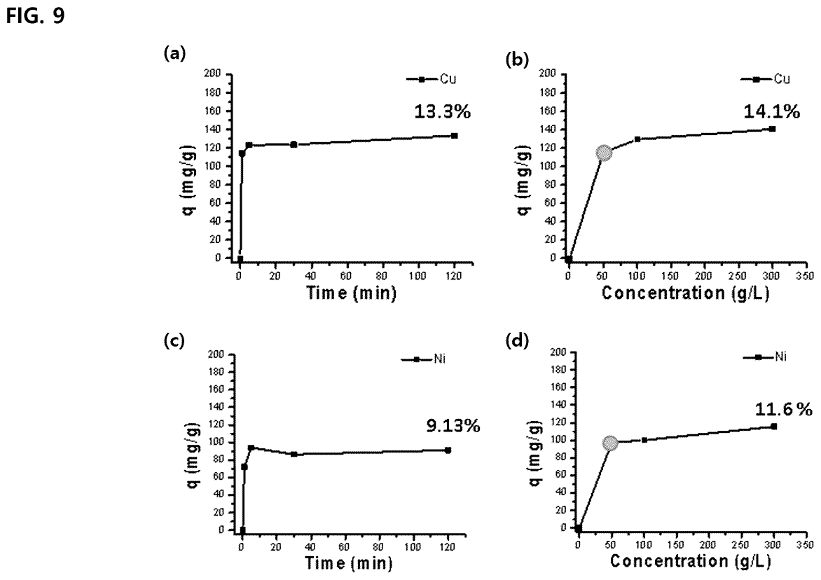

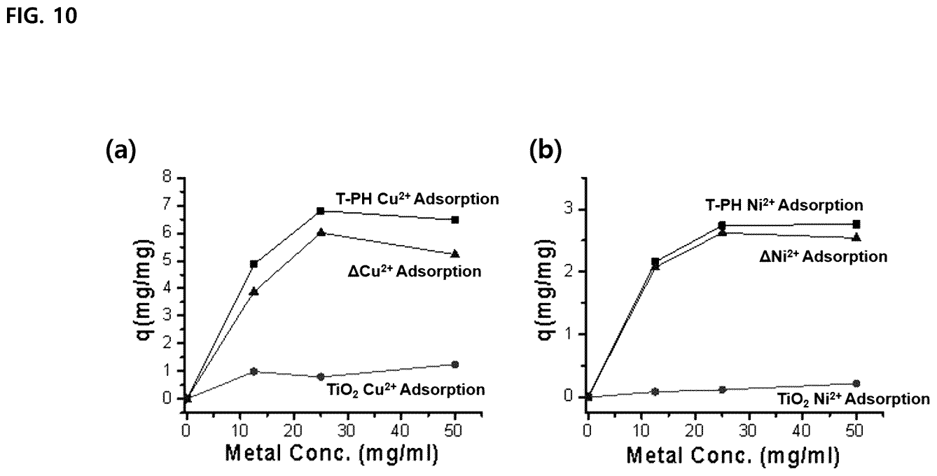

The results are shown in FIGS. 9 and 10.

As shown in FIG. 9 showing graphs illustrating the heavy metal adsorption capacity of Material 2 (PH), the amounts of adsorption increased over time.

As shown in FIG. 10, compared with titanium dioxide (TiO₂) having only an ultraviolet-blocking effect, the titanium dioxide (TiO₂) composite particles with the heteroatomic compound further attached thereto (Material 3, T-PH) had excellent heavy metal adsorption capacity.

Resultantly, the titanium dioxide (TiO₂) composite particles with the heteroatomic compound further attached thereto (Material 3, T-PH) of the present disclosure exhibited not only an ultraviolet-blocking effect but also an excellent heavy metal adsorption effect.

### Test Example 1-3: White Cast Reducing Effect

To evaluate an effect of mitigating the white cast, a typical disadvantage shown when titanium dioxide (TiO₂) is used in the composition of a cosmetic product, the titanium dioxide (TiO₂) composite particles (Material 1, T-PSI) prepared in Preparation Example 1-1 and the titanium dioxide (TiO₂) composite particles with the heteroatomic compound further attached thereto (Material 3, T-PH) were measured for whiteness index by using a colorimeter.

The results are shown in Table 1.

TABLE 1

| Classification | Whiteness index |
|---|---|
| Cream | 47.03 |
| TiO₂ | 46.51 |
| T-PSI | 41.04 |
| T-PH-21 | 35.14 |

As shown in Table 1, compared with titanium dioxide (TiO₂), Material 1 (T-PSI) and Material 3 (T-PH) showed reduced whiteness indexes, indicating a reduced white cast.

### Test Example 1-4: Verification of Photo-Catalytic Property Reducing Effect

The use of titanium dioxide (TiO₂) in the cosmetic composition may cause skin oxidation due to the photo-catalytic properties of TiO₂. Hence, the titanium dioxide (TiO₂) composite particles (Material 1) prepared in Preparation Example 1-1 and the titanium dioxide (TiO₂) composite particles with the heteroatomic compound further attached thereto (Material 3) were investigated for a photo-catalytic property reducing effect.

Each titanium dioxide composite particle material (0.1 g) was added to 100 ml of a 10 ppm methylene blue solution, followed by exposure to 254 nm UV light (24 W), and then the photo-catalytic property effect of the titanium dioxide (TiO₂) composite particle material was compared while the degree of reduction in methylene blue absorbance was verified at a wavelength of 666 nm.

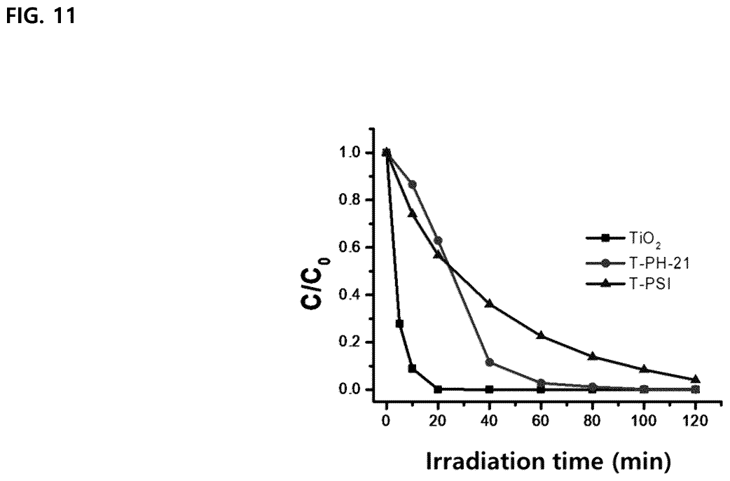

The results are shown FIG. 11.

As shown in FIG. 11, the titanium dioxide (TiO₂) composite particles (Material 1) prepared in Preparation Example 1 and the titanium dioxide (TiO₂) composite particles with the heteroatomic compound further attached thereto (Material 3) showed lower photo-catalytic effects than titanium dioxide (TiO₂).

### Preparation Examples 2

### Preparation Example 2-1. Preparation of Biocompatible Polyamino Acid Polymer Material (Material 4, PA) Having Heavy Metal Adsorption Capacity

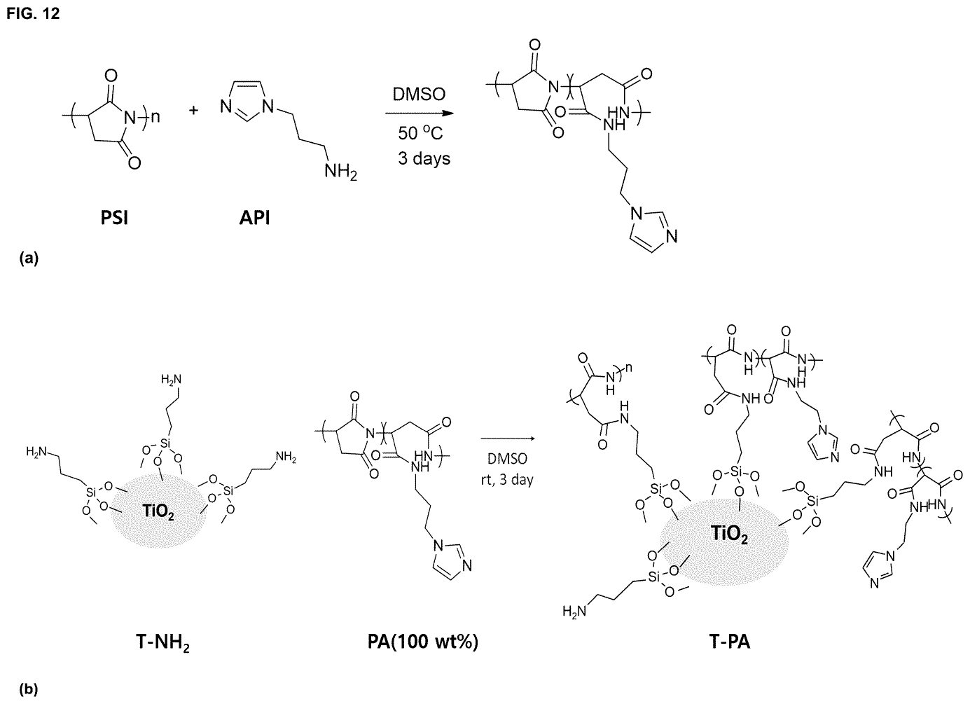

A biocompatible polyamino acid polymer material (Material 4) having heavy metal adsorption capacity was prepared by attaching a heteroatomic compound to polysuccinimide (PSI), a polyamino acid biocompatible polymer material. A schematic diagram of the reaction is shown in FIG. 12.

Specifically, PSI (1 g) was added into 30 ml of DMSO and dissolved with sufficient stirring. Thereafter, 1-(3-Aminopropyl) imidazole (0.36 mL) was followed by stirring at room temperature for 24 h. Then, the product was obtained by drying through vacuum. The yield was 99%.

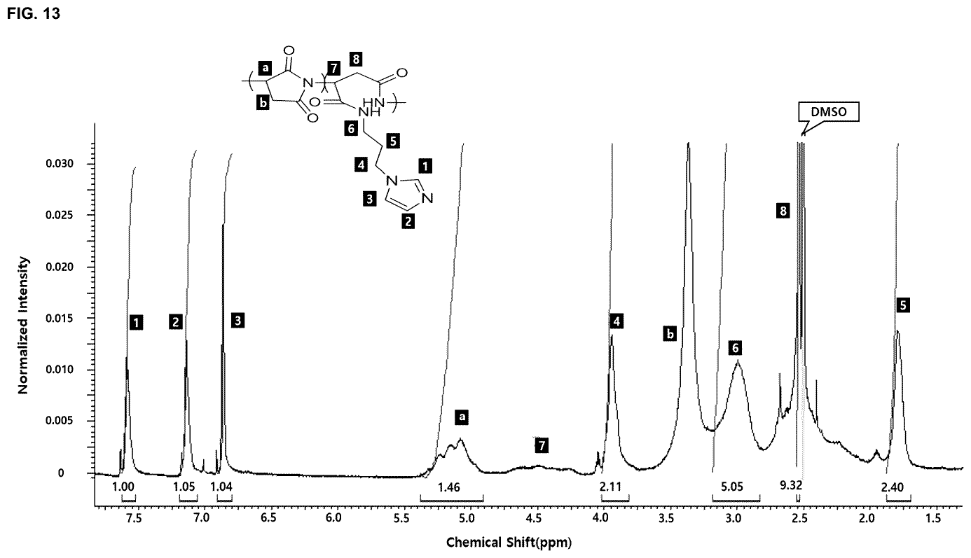

NMR data for Material 4 (PA) are shown in FIG. 13. FIG. 13 confirmed the synthesis of Material 4.

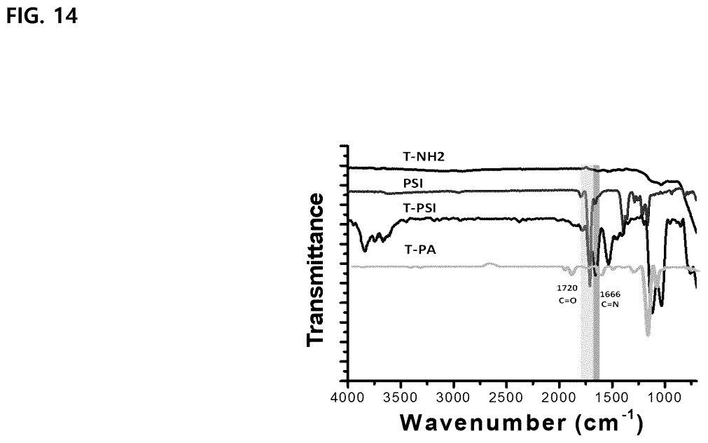

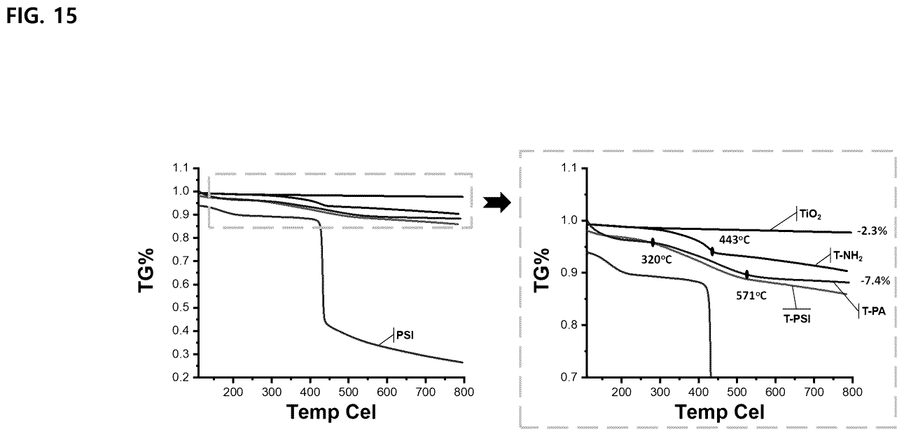

In addition, FT-IR data and TGA data for Material 4 (PA) are shown in FIGS. 14 and 15, respectively.

### Preparation Example 2-2. Preparation of Titanium Dioxide (TiO₂) Composite Particles (Material 5, T-PA) Having Ultraviolet-Blocking Properties and Heavy Metal Adsorption Capacity

The TiO₂ derivative (T-NH₂) of Preparation Example 1-1 and the biocompatible polyamino acid polymer material (PA) having heavy metal adsorption capacity of Preparation Example 2-1 were reacted to produce titanium dioxide (TiO₂) composite particles (Material 5, T-PA) having both ultraviolet-blocking properties and heavy metal adsorption capacity.

Specifically, the TiO₂ derivate (T-NH₂) (2 g) modified with the amine group was added to 40 ml of DMSO, followed by ultrasonic treatment for 30 min. Thereafter, 100 wt % of PA (Material 4) was added, followed by stirring at room temperature for 24 h. The product was obtained by centrifugation, and the yield was 70.0%.

FT-IR data and TGA data for Material 5 (T-PA) are shown in FIGS. 14 and 15, respectively.

### Test Examples 2

### Test Example 2-1. Verification of Ultraviolet-Blocking Effect

To verify the UV blocking effects of the titanium dioxide (TiO₂) composite particles (Material 1, T-PSI) prepared in Preparation Example 1-1 and the titanium dioxide (TiO₂) composite particles with the heteroatomic compound further attached thereto (Material 5, T-PA), UV-Vis spectrum analysis was performed to evaluate the absorbance and transmittance.

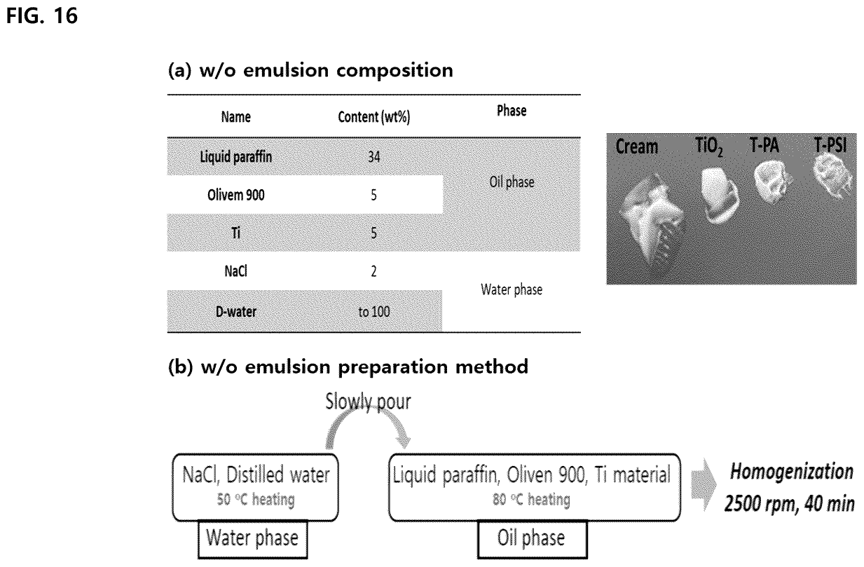

In this example, powdery TiO₂ confirmed to have an ultraviolet-blocking effect was used as a control group. By using (a) the composition of the water-in-oil (w/o) emulsion and (b) the preparation method thereof as shown in FIG. 16, each material was coated in a uniformly mixed cream form and was coated in a cream form on a polymethyl methacrylate (PMMA) plate to evaluate the ultraviolet-blocking effect.

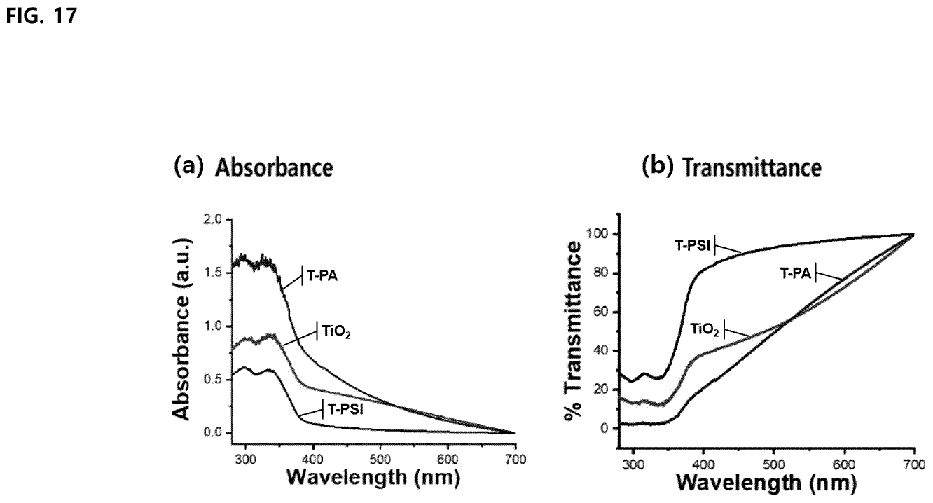

The results are shown (a) and (b) of FIG. 17.

As shown in (a) and (b) of FIG. 17, Material 1 (T-PSI) and Material 5 (T-PA) of the present disclosure exhibited an ultraviolet-blocking effect at a similar level to the powdery TiO₂ used as a control group.

### Test Example 2-2. Verification of Heavy Metal Adsorption Effect

To verify the heavy metal adsorption effect of the biocompatible polyamino acid polymer material (Material 4, PA) prepared in Preparation Example 2-1 and the titanium dioxide (TiO₂) composite particles with the heteroatomic compound further attached thereto (Material 5, T-PA), inductively coupled plasma (ICP) analysis was performed.

Specifically, Material 4 (PA) was exposed to copper (Cu²⁺) and nickel (Ni²⁺) aqueous solutions for 5 min, 30 min, and 2 h, respectively, and the adsorbed materials were re-precipitated in methanol (MeOH) two times. After that, the amounts of heavy metals adsorbed were measured to verify the heavy metal adsorption capacity. To measure the degrees of adsorption of copper (Cu²⁺) and nickel (Ni²⁺) depending on three concentrations (50,000 mg/L, 100,000 mg/L, and 300,000 mg/L), Material 4 was exposed to metal solutions with the three concentrations for 12 h, and then the adsorbed materials were re-precipitated in methanol two times to re-elute un-adsorbed heavy metals. After that, the amounts of heavy metals adsorbed were measured to verify the heavy metal adsorption capacity.

Material 5 was exposed to copper (Cu²⁺) and nickel (Ni²⁺) aqueous solutions for 12 h, and then the adsorbed materials were washed with water three times to re-elute un-adsorbed heavy metals. After that, the amounts of heavy metals adsorbed were measured to verify the heavy metal adsorption capacity.

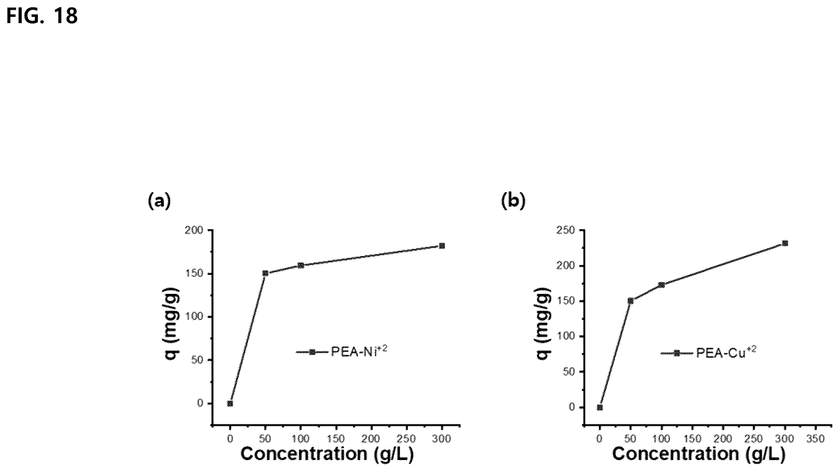

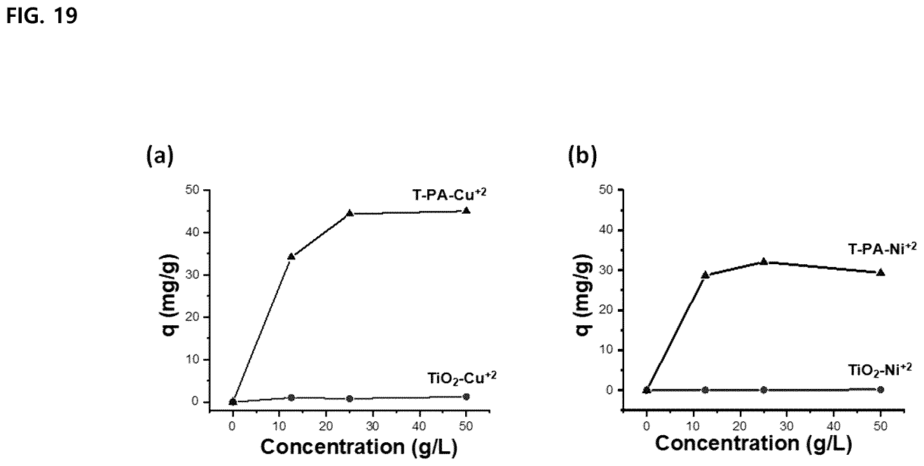

The results are shown in FIGS. 18 and 19.

As shown in FIG. 18 showing graphs illustrating the heavy metal adsorption capacity of Material 4 (PA), the amounts of adsorption increased over time.

As shown in FIG. 19, compared with titanium dioxide (TiO₂) having only an ultraviolet-blocking effect, the titanium dioxide (TiO₂) composite particles with the heteroatomic compound further attached thereto (Material 5, T-PA) had excellent heavy metal adsorption capacity.

Resultantly, the titanium dioxide (TiO₂) composite particles with the heteroatomic compound further attached thereto (Material 5, T-PA) of the present disclosure exhibited not only an ultraviolet-blocking effect but also an excellent heavy metal adsorption effect.

### Test Example 2-3: White Cast Reducing Effect

To evaluate an effect of mitigating the white cast, a typical disadvantage shown when titanium dioxide (TiO₂) is used in the composition of a cosmetic product, the titanium dioxide (TiO₂) composite particles (Material 1, T-PSI) prepared in Preparation Example 1-1 and the titanium dioxide (TiO₂) composite particles with the heteroatomic compound further attached thereto (Material 5, T-PA) were measured for whiteness index by using a colorimeter.

The results are shown in Table 2.

TABLE 2

| Classification | Whiteness index |
|---|---|
| Basic Cream | 47.03 |
| TiO₂-21 | 52.26 |
| T-PSI-21 | 41.04 |
| T-PA-21 | 36.21 |

As shown in Table 2, compared with titanium dioxide (TiO₂), Material 1 (T-PSI) and Material 5 (T-PA) showed reduced whiteness indexes, indicating a reduced white cast.

### Test Example 2-4: Verification of Photo-Catalytic Property Reducing Effect

The use of titanium dioxide (TiO₂) in the cosmetic composition may cause skin oxidation due to the photo-catalytic properties of TiO₂. Hence, the titanium dioxide (TiO₂) composite particles (Material 1, T-PSI) prepared in Preparation Example 1-1 and the titanium dioxide (TiO₂) composite particles with the heteroatomic compound further attached thereto (Material 5, T-PA) were investigated for a photo-catalytic property reducing effect.

Each titanium dioxide composite particle material (0.1 g) was added to 100 ml of a 10 ppm methylene blue solution, followed by exposure to 254 nm UV light (24 W), and then the photo-catalytic property effect of the titanium dioxide (TiO₂) composite particle material was compared while the degree of reduction in methylene blue absorbance was verified at a wavelength of 664 nm.

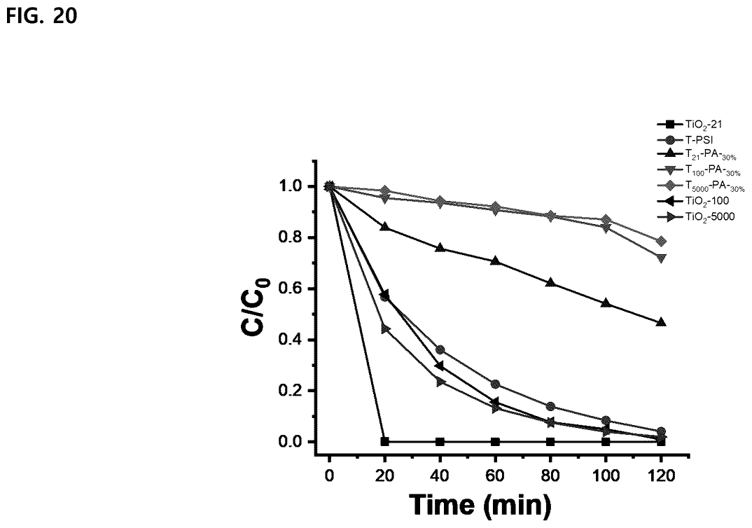

The results are shown FIG. 20.

As shown in FIG. 20, the titanium dioxide (TiO₂) composite particles (Material 1, T-PSI) prepared in Preparation Example 1 and the titanium dioxide (TiO₂) composite particles with the heteroatomic compound further attached thereto (Material 5, T-PA) showed lower photo-catalytic effects than titanium dioxide (TiO₂).

1. A method for preparing titanium dioxide composite particles, the method comprising:
(a) preparing a titanium dioxide (TiO₂) derivative with an amine (—NH₂) functional group introduced to the surface of titanium dioxide (TiO₂); and
(b) reacting the titanium dioxide (TiO₂) derivative obtained in step (a) and a polyamino acid derivative to fuse the titanium oxide derivative and the polyamino acid derivative.

2. The method of claim 1, further comprising, before step (b), attaching a heteroatomic compound to the polyamino acid derivative.

3. The method of claim 1, wherein the titanium dioxide (TiO₂) derivative with the amine functional group is formed by reaction of titanium dioxide and an aminosilane at a weight ratio of 1:1 to 1:5.

4. The method of claim 1, wherein the polyamino acid derivative is at least one selected from the group consisting of polysuccinimide, polyaspartic acid, polyaspartamide, and polyhydroxyethyl aspartamide.

5. The method of claim 1, wherein the polyamino acid derivative has a heteroatomic compound further attached thereto.

6. The method of claim 5, wherein the heteroatomic compound is at least one selected from the group consisting of histamine, aminoimidazole, aminomethylimidazole, aminopropylimidazole, aminomethylbenzimidazole, and histidine.

7. A titanium dioxide composite particle represented by Chemical Formula 1 below: 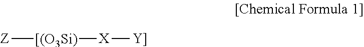
Z is a titanium dioxide particle;
X is a C₁ to C₆ linker; and
Y is 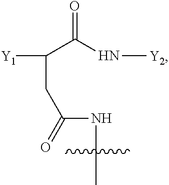
where Y, and Y, each are independently Compound A, Compound B, or a copolymer of Compound A and Compound B.
wherein Compound A is 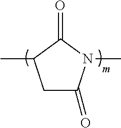
and Compound B is 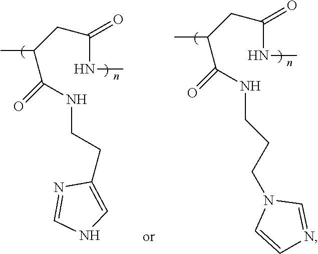
where m and n each are independently an integer of 1 to 1500.

8. The particle of claim 7, wherein in the copolymer of Compound A and Compound B, Compound A and Compound B are present in a random sequence.

9. The particle of claim 7, wherein in the copolymer of Compound A and Compound B, Compound A and Compound B are present in an alternating sequence.

10. The particle of claim 7, wherein Y has the following structure: 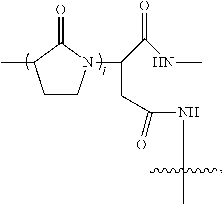
where l is an integer of 0 to 1500.

11. The particle of claim 7, wherein Y has the following structure: 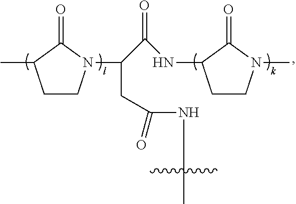
where l and k each are independently an integer of 0 to 1500.

12. The particle of claim 7, wherein Y has the following structure: 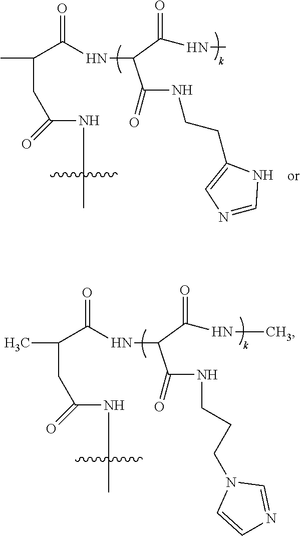
wherein k is an integer of 1 to 800.

13. The particle of claim 7, wherein Y has the following structure: 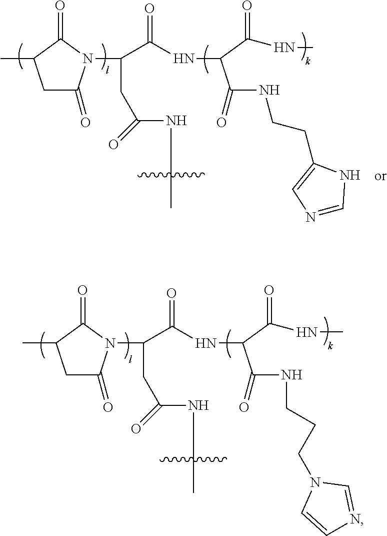
where l and k each are independently an integer of 1 to 1500.

14. A polyamino acid polymer comprising Compound A and Compound B as monomers,
wherein Compound A is 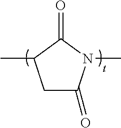
and Compound B is 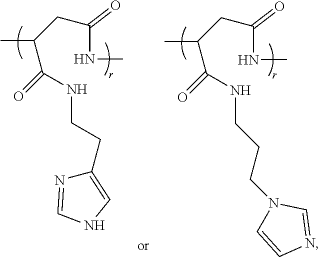
where t and r each are independently an integer of and 1 to 1500.

15. The polymer of claim 14, wherein Compound A and Compound B are present in a random sequence.

16. The polymer of claim 14, wherein Compound A and Compound B are present in an alternating sequence.

17. An ultraviolet-blocking cosmetic composition containing the titanium dioxide composite particle of claim 7.

18. An anti-pollution cosmetic composition containing the polyamino acid polymer of claim 14.
